(19) United States
(12) Patent Application Publication
(10) Pub. No.: US 2009/0069737 A1
(43) Pub. Date: Mar. 12, 2009

(54) Pressure relief dressing

(76) Inventors: Scott Stapley, Portland, OR (US); Renee LaChance, Portland, OR (US)

Correspondence Address: SCOTT STAPLEY, 1731 NE 10TH AVE., PORTLAND, OR, 97212, US

(21) Appl. No.: 11/725,428

(22) Filed: Sep. 6, 2007

(51) Int. Cl. A61F 13/02

(52) U.S. Cl. 602/46; 602/54

(57) ABSTRACT

The Pressure Ulcer Bandage is designed for application to the skin of an individual to treat pressure ulcers. The bandage will be constructed of flexible, round hydrocellular polyurethane foam, approximately one inch deep and will range in diameter with a precut opening to allow pressure relief from the ulcer itself and disperse weight away from the wound. The precut openings should be in various sizes to allow an opening at least 0.25 inches larger than the wound to be utilized The foam bandage will have at least 1.5 times the size of the precut opening of remaining foam outside the opening. Ring shaped bandages have been ineffective in the past because of the lack of flexibility of the material used and the limited amount of padding away from the opening. This bandage can be produced in an adhesive and in a non-adhesive format. Additionally, the packaged product will be sterile.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

A pressure ulcer is an area of skin that breaks down when a person stays in one position for too long without shifting their weight. This often happens if they use a wheelchair or are bedridden, even for a short period of time. The constant pressure against the skin reduces the blood supply to that area, and the affected tissue dies.

A pressure ulcer starts as reddened skin but gets progressively worse, forming a blister, then an open sore, and finally a crater.

Current methods of wound treatment are inadequate for individuals confined to a wheelchair or bedridden. Pressure ulcers are a chronic problem for these individuals because they can never get pressure fully off the wound. For active individuals confined to a wheelchair transferring from the chair to other locations can cause the constant reopening of the wound and further hinder healing.

This pressure ulcer bandage was specifically designed and tested for those confined to a wheelchair who develop pressure ulcers, though the concept can be utilized for other areas of the body and individuals as well.

### REFERENCES

Brillhart B. Pressure sore and skin tear prevention and treatment during a 10-month program. Rehabil Nurs. 2005; 30(3): 85-91.
de Laat E H, Scholte op Reimer W J, van Achterberg T. Pressure ulcers: diagnostics and interventions aimed at wound-related complaints: a review of the literature. J Clin Nurs. 2005; 14(4): 464-472.
Cole L, Nesbitt C. A three year multiphase pressure ulcer prevalence/incidence study in a regional referral hospital. Ostomy Wound Manage. 2004; 50(11): 3240.

### BRIEF SUMMARY OF THE INVENTION

A flexible, round, foam bandage made from a material with the integrity of a hydrocellular polyurethane dressing, for application to the skin of an individual to treat pressure ulcers. Said bandage will provide full pressure relief around the ulcer site, thus, allowing maximum blood circulation for uninhibited healing to occur.

The bandage will have precut openings where the wound is positioned, an opening at least 0.25 inches larger than the wound should be utilized. With foam, a minimum of 1.5 times the diameter of the opening, remaining beyond the opening to allow for the dispersement of weight away from the wound site to prevent secondary wounds. The bandage will be one inch thick to eliminate contact to the wound and help to disperse pressure.

### BRIEF DESCRIPTION OF THE SEVERAL VIEWS OF THE DRAWING

FIG. 1: circular bandage provided in array of sizes with a precut center hole where the wound will be positioned. An opening at least 0.25 inches larger than the wound should be utilized Minimum 1 inch deep and 1 inch of foam remaining from opening.

FIG. 2: circular bandage provided in array of sizes, with a precut center hole where the wound will be positioned with adhesive sufficient to hold the bandage in place. Minimum 1 inch deep and 1 inch of foam remaining from opening.

### DETAILED DESCRIPTION OF THE INVENTION

A flexible, round, foam bandage made from a material with the integrity of a hydrocellular polyurethane dressing, for application to the skin of an individual to treat pressure ulcers. Said bandage will provide full pressure relief around the ulcer site, thus, allowing maximum blood circulation for uninhibited healing to occur.

Hydrocellular polyurethane dressings are already being manufactured. The modification is that the pressure ulcer bandage will have precut openings where the wound is positioned so no pressure is applied to the wound; an opening at least 0.25 inches larger than the wound should be utilized; remaining foam, a minimum of 1.5 times the diameter of the opening, will reach beyond the opening to allow for the dispersement of weight away from the wound site to prevent secondary wounds; the pressure ulcer bandage will be one inch thick to eliminate contact to the wound and help to disperse pressure from the wound site.

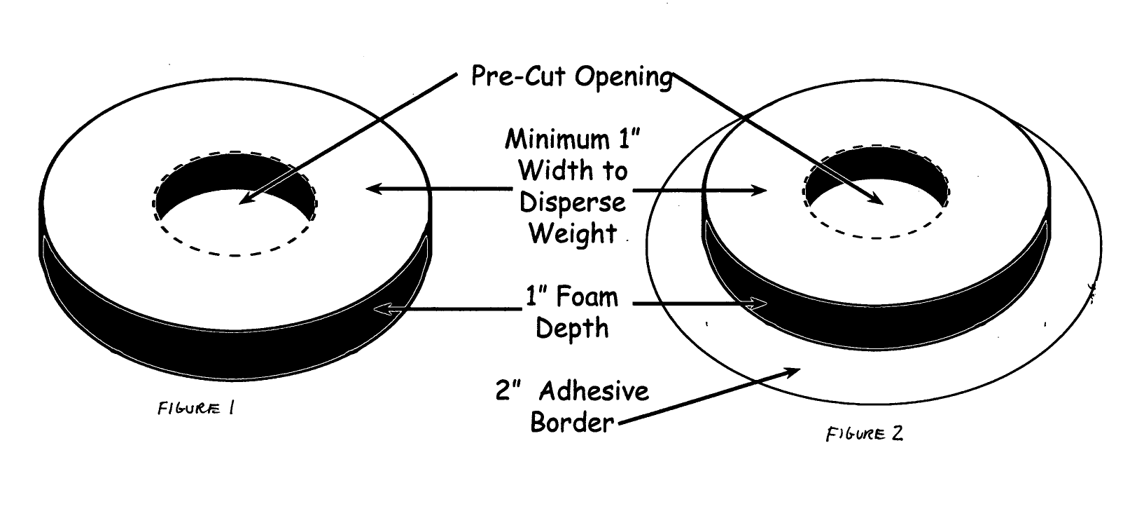

The precut opening keeps pressure off the wound and allows the application of the necessary wound treating protocol without adding pressure to the wound. The flexibility of the hydrocellular polyurethane foam keeps the wound site from developing secondary ulcers by eliminating friction and allowing blood flow to the site. Having sufficient foam remaining around the opening disperses pressure evenly and allows continued circulation to the site and limits complications. (See FIGS. 1 and 2 of drawing.)

What is claimed is:

1. A flexible round foam bandage of material of at least the integrity of a hydrocellular polyurethane dressing for application to the skin of an individual to treat pressure ulcers that will provide full pressure relief from the ulcer site, thus, allowing full circulation for uninhibited healing to occur:

2. The circular bandage according to claim 1 be provided in an array of sizes, ranging from a 3 inch diameter to approximately 7 inches in diameter with a precut center hole where the wound will be positioned.

3. The bandage in claim 2 will have a precut opening with a minimum of 1 inch foam bandaging remaining beyond the opening to allow for the dispersement of weight away from the wound site.

4. The bandage of claim 1 will be approximately 1 inch in depth to ensure the elimination of contact to the wound.

5. The bandage of claim 1 will be offered with an adhesive border and without an adhesive border.

6. The adhesive border of claim 5 will be a minimum of 2 inches to ensure there is an adequate adhesive border to secure the bandage from the top of the bandage.

7. The bandage in claim 1 will be provided sterile when sealed.
